(19) United States
(12) Patent Application Publication
(10) Pub. No.: US 2018/0318555 A1
(43) Pub. Date: Nov. 8, 2018

(54) Smart Attachment for Urinary Catheters

(71) Applicant: Matthew James Antony Brink, Baltimore, MD (US)

(72) Inventor: Matthew James Antony Brink, Baltimore, MD (US)

(21) Appl. No.: 15/972,219

(22) Filed: May 6, 2018

Related U.S. Application Data
(60) Provisional application No. 62/503,264, filed on May 8, 2017.

(51) Int. Cl. A61M 25/04; A61M 25/00

(52) U.S. Cl. CPC A61M 25/04; A61B 5/205; A61M 25/0017

(57) ABSTRACT

The device described in this document provides a method for easily converting a urinary catheter to one that will monitor pressure changes in the anchoring balloon allowing safe deflation if a patient intentionally or unintentionally pulls on the catheter with such force that would otherwise cause traumatic injury to the urethra. The device would sound an alarm in such cases of forced removal. The device would secondarily allow monitoring of insertion date, and intraabdominal pressures among other things due to the electronic storage, display, and communication capabilities.

### CROSS REFERENCE TO RELATED APPLICATION

This utility application corresponds to Provisional Application No. 62/503,264, filed May 8, 2017, entitled “Foley Catheter Safety and Monitoring Device.” Applicant hereby claims the benefit of the filing date of the Provisional Application.

### TECHNICAL FIELD

This disclosure relates to improving urinary catheters that have a balloon anchor.

### BACKGROUND

Urinary catheters are commonly used to drain urine from the urinary bladder. The catheter is secured in place by inflating a balloon at its end with water after it has been inserted into the urethra. Unfortunately, this presents a danger to the patient if they are prone to pulling the catheter out while the balloon is inflated. This can cause bleeding, scarring of the urethra, and infection. In a patient who is, for example, schizophrenic, demented or has an altered mental state, he or she might continuously pull out their urinary catheter causing trauma. This problem is particularly relevant in the elderly for whom dementia and urinary retention are often comorbidities.

Similar inventions have been proposed. One such invention provided a pressure relief system to prevent traumatic removal where the balloon would passively deflate if the catheter was pulled. Other systems have a balloon that breaks with increased pressure caused by pulling on the urinary catheter. Neither of these inventions attach to an existing urinary catheter to provide these safety features. Additionally, the forces involved would be different from patient to patient due to differing anatomy so that a system with an adjustable threshold and a way to safely eject the fluid from the anchoring balloon would be very useful.

Below is a bibliography of illustrative literature in the field of catheters currently known to the applicant. Applicant reserves the right to establish that any of the documents below is not prior art and/or relevant prior art.

### BIBLIOGRAPHY

### Patent Literature

Application US20080103408A1 Marshall T. Denton Wolfe Tory Medical, Inc. Priority 2004 Dec. 3 • Filing 2005 Nov. 21 • Publication 2008 May 1

Application US20160361516A1 Jayson Aydelotte The Board Of Regents Of The University Of Texas System Priority 2012 Jan. 18 • Filing 2016 Mar. 3 • Publication 2016 Dec. 15

Application US20130197486A1 David Aaronson The Regents Of The University Of California Priority 2010 Jul. 9 • Filing 2013 Jan. 9 • Publication 2013 Aug. 1

Grant US5336195A Yousef Daneshvar Yousef Daneshvar Priority 1992 Mar. 19 • Filing 1992 Mar. 19 • Grant 1994 Aug. 9 • Publication 1994 Aug. 08-0

Grant US5429620A Richard C. Davis Uroquest Corporation Priority 1994 Aug. 3 • Filing 1994 Aug. 3 • Grant 1995 Jul. 4 • Publication 1995 Jul. 4

Application US20140012235A1 Leonard Pinchuk Leonard Pinchuk Priority 2006 Jan. 25 • Filing 2013 Sep. 11 • Publication 2014 Jan. 9

### Non-Patent Literature

Prevention of Inappropriate Self-Extraction of Foley Catheters. Leslie S W, Shenot P J. StatPearls [Internet]. Treasure Island (FL): StatPearls Publishing; 2018 January-2018 Mar. 20.

### SUMMARY

A smart catheter attachment in accordance with one example of the invention comprises a mechanism adapted to connect the attachment to a fluid injection port on a urinary catheter, the fluid injection port being in communication with an anchor bag on a distal end of the catheter. The attachment further comprises a signal processor responsive to telemetry from the anchor bag to produce output signals related to conditions in the anchor bag.

In a more detailed example of the invention, this invention would create a safety system for urinary catheters by combining the catheter with an alarm and a pressure transducer. A special fluid ejection pump would trigger the safe release of water from the inflatable anchoring balloon if a patient tried to pull the catheter out. A benefit of attaching such a transducer is that it could also serve as a way to for measure intra-abdominal pressure (a similar technique is used to monitor for intra-abdominal compartment syndrome), and it could allow for an alarm system either to trigger at the bedside or at a nursing station. Having a urinary catheter with a pressure transducer, alarm, and safety valve that could be attached to the inflation port of a traditional urinary catheter would be a potentially life-saving device for this type of patient who could otherwise suffer from scarring, bleeding, urinary obstruction, kidney damage, infection or other complications.

### BRIEF DESCRIPTION OF THE DRAWINGS

FIG. 1 Is a front view of an example of a smart attachment for a urinary catheter in accordance with the invention.

FIG. 2 shows the smart attachment of FIG. 1 connected to an illustrative urinary catheter.

FIG. 3 shows in outline a schematic diagram of the internal components of the attachment of FIG. 1.

FIG. 4 is a front view of an example of a smart catheter attachment in accordance with the invention.

FIG. 5 is a top view of the device shown in FIG. 4.

FIG. 6 is a side view of the device shown in FIG. 4

### DETAILED DESCRIPTION

This description will focus on an illustrative application of the principles of this invention to patients who deliberately or inadvertently pull out a urinary catheter. This will cause inevitable damage to their urethra if the anchoring balloon is not deflated in a rapid and safe way.

The problem would be solved by attaching a device that would act as a pressure transducer, fluid ejection device, and alarm system that would safely eject fluid from the anchoring balloon allowing the urinary catheter to come out automatically. The device would achieve this by monitoring for spikes in pressure caused by pulling on the device, and these thresholds would be adjustable to suit each specific patient's anatomy. The device would also trigger an alarm, which could alert caretakers to the problem. The device would attach to an existing urinary catheter by a self-sealing mechanism that would attach to an existing part of the urinary catheter. The electronic nature of the device, which would be battery powered, would allow for display functionality and for settings to be viewed and adjusted. The electronic component of the device would also allow multiple other functions (such as insertion date, pressure history, urinary output, and other parameters to be measured and stored on the device). A wireless communication component would allow easy integration with existing systems for round-the-clock monitoring of the urinary catheter's status.

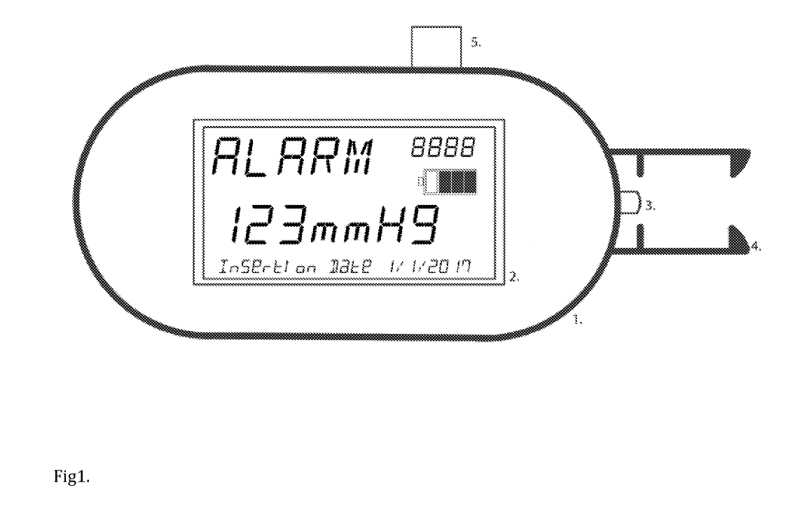

FIG. 1 is a front view of an illustrative safety device for a urinary catheter in accordance with the invention. The device has a housing 1 that would be formed from a light, hard, durable, and waterproof material like plastic. The housing 1 would contain the internal components of the device in a protective shell that would keep the internal components in a fixed position. The display screen 2 would consist of a small liquid crystal or similar display with a light emitting diode or similar lighting source for backlighting. The display screen 2 would preferably be high resolution and capable of color display, and it would have touchscreen capability for accessing control and display functions. The attachment mechanism would include a tube 3 that would mimic the tip of a plastic syringe of the type that is typically used to inflate the anchoring balloon of a urinary catheter with water. The tube 3 would be about 2 mm to 3 mm in external diameter and 1 mm to 2 mm in internal diameter. The tube 3 would be about 5 mm in length. The device is attached by a clasping mechanism 4 that would grasp the plastic fluid-injection port of an existing urinary catheter. The device would have a fluid ejection port 5 that would be about 3 mm in internal diameter where fluid would be ejected from the anchoring balloon of the urinary catheter through a pump mechanism contained within the device housing 1. The ejection port 5 would be made from plastic or a similar material.

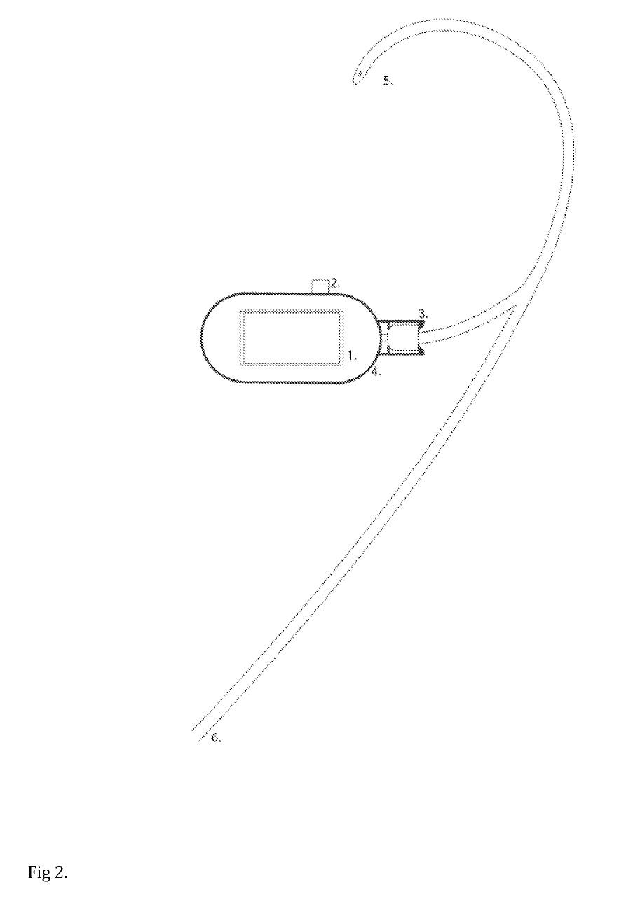

FIG. 2 is a view of the device pictured in FIG. 1 attached to a urinary catheter (the existing urinary catheter is not part of the invention, and is shown for illustrative purposes) as it would be attached during normal use. The display screen is again illustrated by 1. The fluid ejection port 2 is shown continuous with the fluid contained within the urinary catheter's secondary lumen at the urinary catheter's distal end. The secondary lumen in the urinary catheter (not shown) is continuous with the anchoring balloon on the distal end of the urinary catheter (shown in the deflated state in this drawing). The secondary lumen of the urinary catheter and the fluid contained therein would be sealed from the outside by an electromechanically controlled valve that would respond to electronic signals which would open the valve, turn on the pump, and drain the fluid through the secondary lumen of the catheter to drain the anchoring balloon at the catheter tip 5. The self-sealing attachment valve and clasp 3 is again illustrated connecting the housing 4 to the urinary catheter. The urinary catheter empties into the collection bag at its distal end via the tube 6.

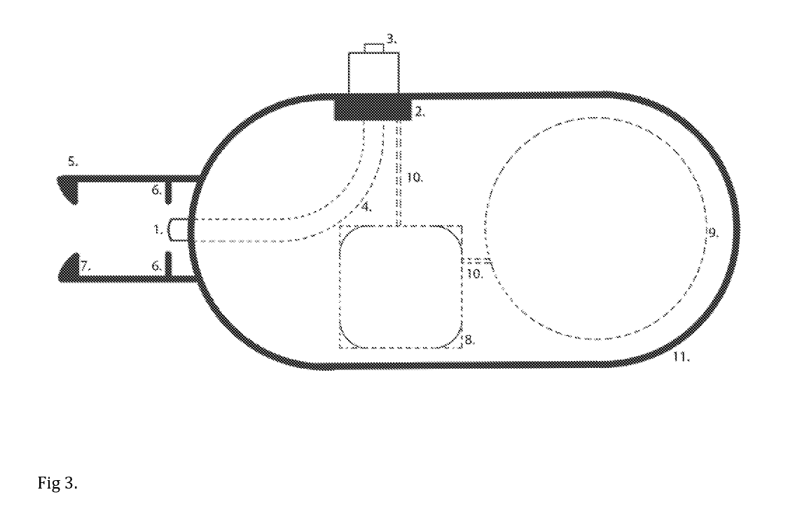

FIG. 3 is a simplified internal view of the device where 11 is the housing of the device which holds the internal components. The tube 3 would be held in a watertight seal with the syringe-tip shaped tube end 1 and the fluid injection port on an existing urinary catheter. The seal would be created with a clasping mechanism containing teeth 7 contained within a tube 5 shown in cross section. The teeth would have a one-way clasping mechanism where the tube 5 and the device housing 11 could be pressed on to the plastic fluid injection port of an existing urinary catheter and held in place by teeth or a ridge 7. There would be a spring mechanism and a valve 6 that would keep a watertight seal with the urinary catheter's secondary lumen via the injection port. The fluid in the secondary lumen continuous with the anchoring balloon at the distal end of the catheter would be continuous with a pressure transducer 2. The pressure transducer would communicate with the computer processing unit 8 which would in turn receive and transmit data wirelessly to a receiver. The CPU 8 would also contain flash memory that would store data and firmware for the device. The computer processing unit 8 would control a miniaturized pump also contained within 2 that would be able to rapidly eject fluid from the anchoring balloon in the urinary catheter via the secondary lumen and then through the device via the tube 4 that leads to the ejection port 3. The internal electrical components of the device would be connected by a printed circuit board or similar connections 10. The CPU 8, the ejection pump and transducer 2, and the electrical components of the device would be powered by a small, flat, compact battery such as a lithium ion battery 9. The lithium ion battery 9 could be charged by induction or by a wired charging port.

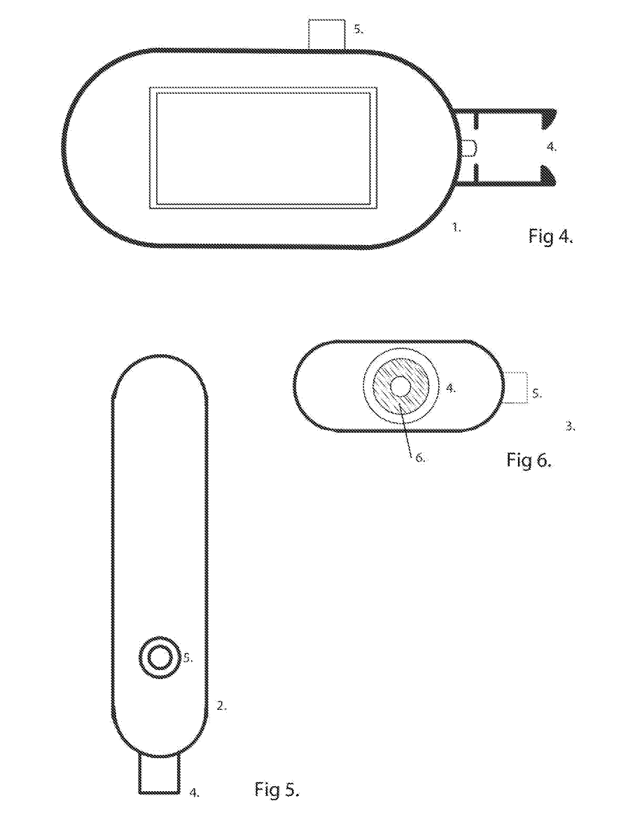

FIG. 4 shows various views of the device where 1 is the anterior view of the housing, 4 is the attachment mechanism, and 5 is the fluid ejection port. A superior view illustrates the ejection port 5 which ejects fluid out of the side of the housing 2. The ejection port 5 is again illustrated in a superior view and a lateral view 3. The lateral view 3 of the attachment mechanism 4 illustrates an axial view of the self-sealing valve 6 which would be pressed against the hard plastic of the urinary catheter's fluid injection port by a spring mechanism which would create a watertight seal.

This invention would potentially stop traumatic urinary catheter removal, which would otherwise result in almost-certain hospitalization and morbidity with potential for life-threatening harm. It would allow easy monitoring of urinary catheters, allowing care staff to reinsert catheters in a timely fashion, to monitor when a catheter needs to be changed (reducing infection risk), among other parameters. This device could effectively create a “smart catheter” from an existing urinary catheter enabling greatly expanded functionality, safety, and convenience for any patient with a urinary catheter. Unlike other inventions that attempt to solve this problem, this device converts existing catheters into “safe catheters” and adds a wealth of “smart function” capability to standard urinary catheters.

This device would be widely useful in the field of inpatient medicine, urology, rehabilitation medicine, geriatric medicine, psychiatry, trauma, and critical care among others. The device could be used in nursing facilities, and even in patient's own homes. It would provide a way to monitor the status of urinary catheters in any patient who requires one, and it would provide greatly expanded functionality. Most importantly, it would prevent some of the major causes of trauma and infection associated with urinary catheters.

### EXAMPLES

### Example 1

The typical use-case scenario for the device would be in a patient who has dementia and coexisting urinary retention that requires an indwelling urinary catheter. The patient would have a urinary catheter (Foley catheter, or similar) inserted by nursing staff and the end bulb would be inflated with water in the usual fashion. The device described above would then be clipped on to the existing inflation port. The device would be secured by a clip that would anchor on to the existing plastic cuff around the inflation port, and the device would create a communicating fluid chamber when the syringe-shaped tip and the surrounding self-sealing valve. The pressure within the urinary catheter anchoring balloon would then be measurable by the transducer within the device. When the device electronically senses a critical rise in pressure, it would trigger the fluid ejection pump that would be designed to extract fluid from the anchoring balloon in a way that is rapid enough to prevent injury but controlled enough that it does not harm surrounding anatomy—these parameters would be programmed in to the CPU and storage components of the device. Once the anchoring balloon is deflated, the urinary catheter would come out harmlessly, and an alarm would be triggered to alert nursing staff that the urinary catheter needs to be replaced.

### Example 2

Another use case scenario for the device would be where it could be attached to a urinary catheter as above, and the date of insertion could be set on the interface. The device would communicate wirelessly and alert care providers when the urinary catheter needs to be changed. This would greatly reduce the rates of infection associated with prolonged use of urinary catheters.

### CONCLUSION

The Title, Technical Field, Background, Summary, Brief Description of the Drawings, Detailed Description, and Abstract are meant to illustrate the preferred embodiments of the invention and are not in any way intended to limit the scope of the invention. The scope of the invention is solely defined and limited in the claims set forth below. It is intended, however, that the claims not be limited to any particular form of mechanical and electronic implementations.

1. A safety device adapted to be attached to a fluid injection port on a urinary catheter, the catheter having an anchoring balloon located on a distal end of the catheter connected to the fluid injection port, comprising:
a pressure sensor adapted to detect fluid pressure in the anchoring balloon; and
an electronic data processor connected to the pressure sensor adapted to produce a control signal in response to a pressure signal from the pressure sensor.

2. The safety device of claim 1, further comprising:
a pump responsive to the control signal to pump fluid from the anchoring balloon when the pressure in the anchoring balloon exceeds a predetermined value.

3. The safety device of claim 1, further comprising:
an alarm responsive to the control signal to report an abnormal pressure in the anchoring balloon.

4. The safety device of claim 1, further comprising:
a temperature sensor responsive to the temperature of the fluid in the catheter to indicate an abnormal temperature in the catheter.

5. The safety device of claim 1, further comprising:
a wireless transceiver adapted to transmit data from the electronic data processor to a communication network.

6. A smart catheter attachment, comprising:
a mechanism adapted to connect the attachment to a fluid injection port on a urinary catheter, the fluid injection port being in communication with an anchor bag on a distal end of the catheter; and
a signal processor responsive to telemetry from the anchor bag to produce output signals related to conditions in the anchor bag.

7. The smart catheter of claim 6, in which the mechanism comprises a self-sealing valve.

8. The smart catheter of claim 7, in which the telemetry comprises fluid pressure in the anchor bag.

9. The smart catheter of claim 7, in which the telemetry comprises fluid temperature in the anchor bag.

10. The smart catheter attachment of claim 8, further comprising:
a pump responsive to a control signal from the signal processor to evacuate fluid from the anchoring balloon when the pressure in the anchoring balloon exceeds a predetermined value.
